(19) United States
(12) Patent Application Publication
(10) Pub. No.: US 2013/0022644 A1
(43) Pub. Date: Jan. 24, 2013

(54) NOVEL N-PHENYLACETAMIDE DERIVATIVES, WHICH INHIBIT THE ENZYME SOAT-1, AND PHARMACEUTICAL AND COSMETIC COMPOSITIONS CONTAINING THEM

(71) Applicants: Jean-Claude Pascal, Nice (FR); Cédric Poinsard, Le Plan de Grasse (FR)

(72) Inventors: Jean-Claude Pascal, Nice (FR); Cédric Poinsard, Le Plan de Grasse (FR)

(73) Assignee: GALDERMA RESEACH & DEVELOPMENT

(21) Appl. No.: 13/202,699

(22) PCT Filed: Feb. 26, 2010
(86) PCT No.: PCT/EP2010/052497

§ 371 Date: Oct. 11, 2011

Related U.S. Application Data
(60) Provisional application No. 61/202,417, filed on Feb. 26, 2009.

(30) Foreign Application Priority Data
Jun. 5, 2009 (FR) 0953754

(51) Int. Cl. A61K 31/4166; A61K 9/00; A61K 8/49; A61K 8/02; A61P 17/08; A61P 17/10; A61P 17/00; A61P 27/02; A61P 3/06; A61P 9/10; A61P 25/28; A61Q 19/00; A61Q 5/00; C07D 233/78; B82Y 5/00

(52) U.S. Cl. 424/400; 548/319.5; 514/389; 977/700; 977/906

(57) ABSTRACT

Compounds of general Formula (I),

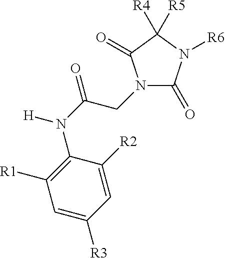

and cosmetic and pharmaceutical compositions including such a compound are described.

The invention relates to novel N-phenylacetamide derivatives, which are inhibitors of the enzyme SOAT-1 (Sterol-O-Acyl Transferase-1, also known as ACAT-1: Acyl-coenzyme A Cholesterol Acyl Transferase). The invention also relates to the use of these derivatives in pharmaceutical compositions intended for use in human or veterinary medicine, or alternatively in cosmetic compositions, and also to their non-therapeutic use.

Compositions with activity of SOAT-1-inhibiting type are widely described in the literature as having activity in regulating biological processes involving cholesterol and derivatives thereof. These properties give this class of compounds strong potential in the treatment or prevention of many pathologies, and more particularly in dermatology and in cardiovascular diseases or central nervous system complaints. Most of the biological effects of SOAT-1 inhibitors are mediated by prevention of the synthesis of cholesterol esters by the enzyme SOAT-1. Among the prior art documents describing SOAT-1-inhibiting molecules, mention may be made, for example, of WO 96/10559, EP 0 370 740, EP 0 424 194, U.S. Pat. No. 4,623,663, EP 0 557 171, U.S. Pat. No. 5,003,106, EP 0 293 880, EP 0 433 662 and U.S. Pat. No. 5,106,873, which describe compounds for treating arteriosclerosis or hypercholesterolaemia. The therapeutic potential of SOAT-1 inhibitors in the treatment of cardiovascular diseases, and in particular of hypercholesterolaemia and arteriosclerosis, is also described by Kharbanda R. K. et al., in Circulation. 2005, 11, 804. The potential of SOAT-1 inhibitors for the treatment of Alzheimer's disease has also been reported in the literature, for example by Puglielli, L. et al., in Nature Neurosciences 2003, 6 (4), 345.

Patents U.S. Pat. No. 6,133,326, U.S. Pat. No. 6,271,268 and WO 2005/034 931 describe SOAT-1-inhibiting compounds for inhibiting the production of sebum. In the field of dermatology, in particular, it is particularly advantageous to prevent excessive sebum production and all the associated conditions. Sebum is produced by the sebaceous glands. The largest concentration of sebaceous glands is found on the face, the shoulders, the back and the scalp. Sebum is secreted at the surface of the skin, where it plays a major physiological role, associated with maintaining the skin barrier and a microenvironment that permits regulation of the cutaneous bacterial and fungal flora.

Sebum hyperproduction is usually associated with a skin or scalp of greasy appearance, which is a cause of discomfort and of degraded appearance. Moreover, sebum hyperproduction may give rise to seborrhoeic dermatitis and is associated with an increased incidence or worsening of acne. The cholesterol esters produced in the sebaceous glands by SOAT-1 are one of the components of sebum, among several classes of lipids including triglycerides, wax esters and squalenes, as described by Nikkari, T., in J. Invest. Derm. 1974, 62, 257. Inhibition of this enzyme or of other acyl transferases may thus make it possible to inhibit sebum production. Patent U.S. Pat. No. 6,133,326 especially describes the inhibition of sebum with ACAT-1 (also known as SOAT-1) inhibitors. However, at the present time, no treatment using such inhibitors is commercially available. The only treatments that can remedy or relieve hyperseborrhoea-related disorders are systemic hormonal treatments or systemic treatment with 13-cis-retinoic acid, the side effects of which treatments greatly limit their field of application. There is thus a clear medical and cosmetic need to treat complaints and pathologies related to sebum hyperproduction.

In this context, the present invention proposes to provide novel N-phenylacetamide derivatives that are powerful inhibitors of the enzyme SOAT-1.

One subject of the invention is novel dioxo-imidazolidine derivatives, which are inhibitors of the enzyme SOAT-1, and which correspond to the general formula (I) below:

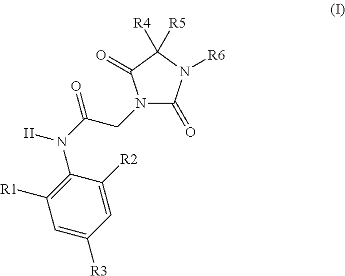

in which:
R₁ represents a halogen, a group C₁₋₆ alkyl, C₃₋₇ cycloalkyl, C₁₋₆ alkyloxy, C₁₋₆ fluoroalkyl or C₁₋₆ fluoroalkyloxy or a group —(CH₂), —C₃₋₇ cycloalkyl,
R₂ and R₃ are identical or different and represent a hydrogen, chlorine, fluorine, bromine or iodine atom or a group C₁₋₆ alkyl, C₃₋₇ cycloalkyl, C₁₋₆ alkyloxy, C₁₋₆ fluoroalkyl or C₁₋₆ fluoroalkyloxy or a group —(CH₂)ₙ—C₃₋₇ cycloalkyl,
R₄ and R₅ are different from each other and individually represent:
   either a hydrogen atom,
   or a group C₁₋₆ alkyl optionally substituted with one to three groups Rₐ,
   or a group C₃₋₇ cycloalkyl or a group —(CH₂)ₙ—C₃₋₇ cycloalkyl,
R₆ represents a group chosen from:
   an unsubstituted phenyl group or a phenyl group substituted with one, two or three identical or different substituents chosen from fluorine, chlorine and bromine atoms and groups C₁₋₄ alkyl, C₁₋₄ alkylthio, trifluoromethyl, hydroxymethyl, mono-, di- and tri-fluoromethoxy, C₁₋₄ alkyloxy, hydroxyl, COOR_{b}, CN, phenoxy, benzyloxy, phenyl, 2-pyridyl, 3-pyridyl and 4-pyridyl,
   a linear or branched group C₂₋₁₂ alkyl, optionally substituted with one or more hydroxyl groups or fluorine atoms,
   a group C₃₋₇ cycloalkyl or a group —(CH₂)ₚ—C₃₋₇ cycloalkyl,
   a group —(CH₂)ₙ-aryl in which n is equal to 1, 2 or 3 and the aryl group may be optionally substituted with one or more groups Rₐ,
   a group —(CH₂)ₙ—Ar with n equal to 1, 2 or 3 and Ar representing an unsubstituted phenyl or unsubstituted or naphthyl group, or a phenyl or naphthyl group substituted with one to three identical or different substituents chosen from fluorine, chlorine, iodine or bromine atoms and groups C₁₋₆ alkyl, hydroxymethyl, mono-, di- or trifluoromethyl, hydroxy, phenyl, 2-pyridyl, 3-pyridyl or 4-pyridyl, C₁₋₆alkyloxy, phenoxy, benzyloxy, and mono-, di- or trifluoromethoxy,

Rₐ represents either a hydrogen, fluorine, chlorine or bromine atom or a group C₁₋₆ alkyl, C₃₋₇ cycloalkyl, C₁₋₆ alkyloxy, C₁₋₆ alkylthio, C₁₋₆ fluoroalkyl or C₁₋₆ fluoroalkyloxy, or a group —(CH₂)ₙ—C₃₋₇ cycloalkyl, OH, COOR_{b}, or CN,
R_{b} represents a group C₁₋₆ alkyl, C₃₋₇ cycloalkyl or —(CH₂)ₙ—C₃₋₇ cycloalkyl,
n is an integer equal to 1, 2 or 3,
   and also the pharmaceutically acceptable salts, solvates or hydrates thereof and the conformers or rotamers thereof.

The compounds of formula (I) may comprise one or more asymmetric carbon atoms. They may thus exist in the form of a mixture of enantiomers or of diastereoisomers. These enantiomers and diastereoisomers, and also mixtures thereof, including racemic mixtures, form part of the invention.

The compounds of formula (I) may exist in the form of bases or of acid-addition salts. Such addition salts form part of the invention. These salts are advantageously prepared with pharmaceutically acceptable acids, but the salts of other acids that are useful, for example for purifying or isolating the compounds of formula (I), also form part of the invention. These acids may be, for example, picric acid, oxalic acid or an optically active acid, for example a tartaric acid, a dibenzoyltartaric acid, a mandelic acid or a camphorsulfonic acid, and those that form physiologically acceptable salts, such as hydrochloride, hydrobromide, sulfate, hydrogen sulfate, dihydrogen phosphate, maleate, fumarate, 2-naphthalenesulfonate or para-toluenesulfonate. For a review of physiologically acceptable salts, see the Handbook of Pharmaceutical Salts: Properties, Selection and Use by Stahl and Wermuth (Wiley-VCH, 2002).

The solvates or hydrates may be obtained directly after the synthetic process, compound (1) being isolated in the form of a hydrate, for example a monohydrate or hemihydrate, or of a solvate of the reaction or purification solvent.

The present invention includes the isotopically labelled pharmaceutically acceptable compounds of formula (I) in which one or more atoms are replaced with atoms having the same atomic number but an atomic mass or a mass number different from the atomic mass or the mass number that naturally predominates. Examples of isotopes that may be included in the compounds of the invention include hydrogen isotopes such as ²H and ³H, carbon isotopes such as ¹¹C, ¹³C and ¹⁴C, chlorine isotopes such as ³⁶Cl, fluorine isotopes such as ¹⁸F, iodine isotopes such as ¹²³I and ¹²⁵I, nitrogen isotopes such as ¹³N and ¹⁵N, oxygen isotopes such as ¹⁵O, ¹⁷O and ¹⁸O, phosphorus isotopes such as ³²P and sulfur isotopes such as ³⁵S. Substitutions with isotopes that emit positrons, such as ¹¹C, ¹⁸F, ¹⁵O and ¹³N, may be useful in Positron Emission Tomography studies for studying the occupation of receptors.

In the context of the invention, the following definitions apply:
C_{b-c} in which b and c may take values from 1 to 6, a hydrocarbon-based chain of b to c carbon atoms, for example C₁₋₆ is a hydrocarbon-based chain that may contain from 1 to 6 carbon atoms,
alkyl: a linear or branched saturated aliphatic group, for example a group C₁₋₆ alkyl represents a linear or branched hydrocarbon-based chain of 1 to 6 carbon atoms, for example a methyl, ethyl, n-propyl, isopropyl, n-butyl, isobutyl, tert-butyl, pentyl or hexyl,
cycloalkyl: an optionally branched, cyclic saturated hydrocarbon-based chain containing from 3 to 7 carbon atoms. By way of example, a group C₃₋₇ cycloalkyl represents a hydrocarbon-based chain of 3 to 7 carbon atoms, for example a cyclopropyl, cyclobutyl, cyclopentyl, cyclohexyl or cycloheptyl,
alkyloxy: a group —O-alkyl,
alkylthio: a group —S-alkyl,
fluoroalkyl: an alkyl group in which one or more hydrogen atoms have been replaced with a fluorine,
fluoroalkyloxy: an alkyloxy group in which one or more hydrogen atoms have been replaced with a fluorine atom.

A preferred group of compounds of formula (I) defined above is a group (A), in which:
R₁ represents a group C₁₋₆ alkyl, C₃₋₇ cycloalkyl, C₁₋₆ alkyloxy, C₁₋₆ fluoroalkyl or C₁₋₆ fluoroalkyloxy or more favourably a chlorine, methyl, ethyl, isopropyl, tert-butyl, cyclopropyl or CH₂-cyclopropyl group and more preferentially R₁ represents a methyl, ethyl, propyl or isopropyl group,
R₂ represents a chlorine or bromine atom, methyl, ethyl, isopropyl or CH₂-cyclopropyl,
R₃ represents a hydrogen atom.

The group (B) of compounds of formula (I), the substituents R₁, R₂, R₃ and R₆ of which are defined above in the general definition of the compounds of formula (I) or in the preferred group (A) and such that the groups R₄ and R₅ are different and represent either a hydrogen atom or a methyl, ethyl, propyl, butyl, isopropyl, cyclopropyl, cyclobutyl or methylenecyclopropyl group and more preferentially such that R₄ is a methyl and R₅ is an ethyl or a propyl, is a preferred group.

The group (C) of compounds of formula (I), the substituents R₁, R₂, R₃, R₄ and R₅ of which are defined above in the general definition of the compounds of formula (I) or in the preferred groups (A) or (B) and such that the group R₆ represents an unsubstituted phenyl group or a phenyl group substituted in the meta or para position with a chlorine, fluorine, methyl or methoxy group, is a particularly preferred group.

The compounds below, and the pharmaceutically acceptable salts, solvates and hydrates thereof and the conformers or rotamers thereof, are particularly preferred:
N-(2,6-diisopropylphenyl)-2-(4-methyl-2,5-dioxo-4-propyl-3-p-tolylimidazolidin-1-yl)-acetamide;
N-(2,6-diisopropylphenyl)-2-(4-methyl-2,5-dioxo-4-pentyl-3-p-tolylimidazolidin-1-yl)-acetamide;
N-(2,6-diethylphenyl)-2-(4-methyl-2,5-dioxo-4-propyl-3-p-tolylimidazolidin-1-yl)acetamide;
2-[3-(4-chlorophenyl)-4-methyl-2,5-dioxo-4-propylimidazolidin-1-yl]-N-(2,6-diisopropyl-phenyl)acetamide;
N-(2,6-diisopropylphenyl)-2-(4-methoxymethyl-4-methyl-2,5-dioxo-3-p-tolylimidazolidin-1-yl)acetamide.

A subject of the invention is also a process for preparing the compounds of general formula (I).

In accordance with the invention, the compounds of formula (I) may be prepared according to the general process described in Scheme 1 below.

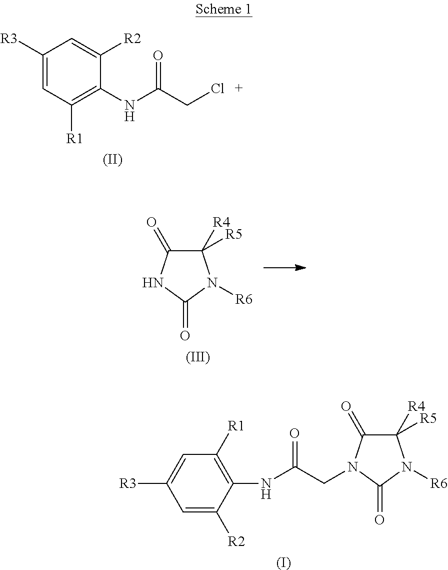

The compounds of formula (I) in which R₁, R₂, R₃, R₄, R₅ and R₆ are as defined above may be prepared by reacting the dioxo-imidazolidines of formula (III) with the chloroacetamides of formula (II), in the presence of a base, according to Scheme 1 and by analogy, for example, with the reactions described by Dunbar, B. et al., Pharmazie 2002, 57 (7), 438, Pinza, M. et al., J. Med. Chem. 1993, 36 (26), 4214, Coudert, P. et al., Pharm. Acta Helv. 1991, 66 (5-6), 155 or Usifoh, C. O.; Arch. Pharm. 2001, 334 (11), 366.

Synthesis of the Intermediates (II) and (III)

The chloroacetamides of general formula (II) may be prepared by reaction between the anilines of formula (VIII) and chloroacetyl chloride in the presence of a base, for example as described in Davion, Y. et al., Heterocycles 2004, 63 (5), 1093 or in Juaristi, E. et al., J. Org. Chem. 1999, 64 (8), 2914, as illustrated in Scheme 2 below in which R₁, R₂ and R₃ are as defined for the compounds of formula (I):

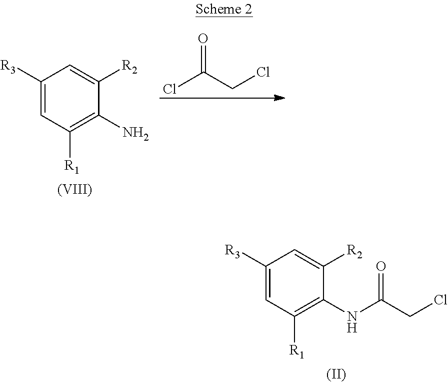

The dioxo-imidazolidines of general formula (III), in which R₄, R₅ and R₆ are as defined above for the compounds of formula (I), may be prepared according to Scheme 3 below:

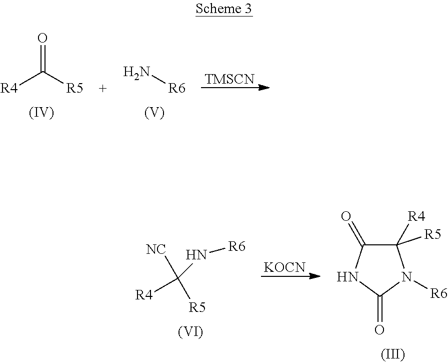

The nitrile compounds of formula (VI) are obtained from the ketones of formula (IV) reacted with the amines of formula (V) in the presence of trimethylsilyl cyanide, in accordance, for example, with the conditions described in Matsumoto K. at al., Helv. Chim. Acta 2005, 88 (7), 1734-1753 or Nieto M. J. et al., J. Comb. Chem. 2005, 7 (2), 258-263.

The ketones (IV) and the amines (V) are commercial compounds or are prepared according to techniques that are well known to those skilled in the art.

The dioxo-imidazolidine intermediates of formula (III) may be prepared by reacting the nitrile derivatives (VI) with potassium isocyanate, followed by work-up in acidic medium according, for example, to the conditions described in patent DE 1 032 258.

The functional groups that may be present in the reaction intermediates used in the process may be protected, either permanently or temporarily, with protecting groups that ensure an unequivocal synthesis of the expected compounds. The protection and deprotection reactions are performed according to techniques that are well known to those skilled in the art. The term “temporary protecting group for amines, alcohols or carboxylic acids” means protecting groups such as those described in “Protective Groups in Organic Chemistry”, published by McOmie J. W. F., Plenum Press, 1973, in “Protective Groups in Organic Synthesis”, 2nd edition, Greene T. W. and Wuts P. G. M., published by John Wiley & Sons, 1991, and in “Protecting Groups”, Kocienski P. J., 1994, Georg Thieme Verlag.

The compounds (1) according to the invention, and also the pharmaceutically acceptable salts, solvates and/or hydrates thereof, have inhibitory properties on the enzyme SOAT-1. This inhibitory activity on the enzyme SOAT-1 is measured according to a HepG2 primary enzymatic test, as described in Example 3. The preferred compounds of the present invention have a concentration that enables inhibition of 50% of the response of the enzyme (IC₅₀) of less than or equal to 1000 nM, preferentially less than or equal to 300 nM and advantageously less than or equal to 50 nM.

A subject of the present invention is also, as medicaments, the compounds of formula (I) as described above, and also the pharmaceutically acceptable salts and pharmaceutically acceptable solvates and/or hydrates thereof.

A subject of the present invention is the use of at least one compound of formula (I), or pharmaceutically acceptable salts or solvates and/or hydrates thereof, for the manufacture of a medicament for preventing and/or treating sebaceous gland disorders such as hyperseborrhoea, acne, seborrhoeic dermatitis or atopic dermatitis, ocular pathologies such as blepharitis or meibomitis (disorder of the Meibomian gland) or pathologies such as hypercholesterolaemia, arteriosclerosis or Alzheimer's disease. The compounds according to the invention are particularly suitable for the manufacture of a pharmaceutical composition for treating acne. The compounds according to the invention are thus suitable for use in the pathologies listed above.

A subject of the present invention is also a pharmaceutical or cosmetic composition comprising, in a physiologically acceptable support, at least one compound of formula (I) as defined above, or a pharmaceutically acceptable salt or solvate and/or hydrate thereof. The compositions according to the invention thus comprise a physiologically acceptable support or at least one physiologically or pharmaceutically acceptable excipient, chosen according to the desired cosmetic or pharmaceutical form and the chosen mode of administration.

The term “physiologically acceptable support or medium” means a support that is compatible with the skin, mucous membranes and/or the integuments.

The administration of the composition according to the invention may be performed via the enteral, parenteral, rectal, topical or ocular route. Preferably, the pharmaceutical composition is conditioned in a form that is suitable for topical application.

Via the enteral route, the composition, more particularly the pharmaceutical composition, may be in the form of tablets, gel capsules, coated tablets, syrups, suspensions, solutions, powders, granules, emulsions, microspheres or nanospheres or lipid or polymer vesicles allowing controlled release. Via the parenteral route, the composition may be in the form of solutions or suspensions for perfusion or for injection.

The compositions according to the invention contain a compound according to the invention, in an amount sufficient to obtain the desired therapeutic, prophylactic or cosmetic effect. The compounds according to the invention are generally administered at a daily dose of about 0.001 mg/kg to 100 mg/kg of body weight, in 1 to 3 dosage intakes. The compounds are used systemically at a concentration generally of between 0.001% and 10% by weight and preferably between 0.01% and 5% by weight relative to the weight of the composition.

Via the topical route, the pharmaceutical composition according to the invention is more particularly intended for treating the skin and mucous membranes and may be in the form of ointments, creams, milks, pomades, powders, impregnated pads, syndets, solutions, gels, sprays, mousses, suspensions, lotions, sticks, shampoos or washing bases. It may also be in the form of suspensions of microspheres or nanospheres or lipid or polymer vesicles or polymer patches and hydrogels allowing controlled release. This topical composition may be in anhydrous form, in aqueous form or in the form of an emulsion.

The compounds are used topically at a concentration generally of between 0.001% and 10% by weight and preferably between 0.01% and 5% by weight relative to the total weight of the composition.

The compounds of formula (I) according to the invention and the pharmaceutically acceptable salts or solvates and/or hydrates thereof also find an application in the cosmetics field, in particular in body and hair hygiene and more particularly for combating or preventing greasy skin or hair or a greasy scalp.

A subject of the invention is thus also the cosmetic use of a composition comprising, in a physiologically acceptable support, at least one of the compounds of formula (I), optionally in the form of a pharmaceutically acceptable salt or solvate and/or hydrate, for body or hair hygiene.

The cosmetic composition according to the invention containing, in a cosmetically acceptable support, at least one compound of formula (I) or a pharmaceutically acceptable salt or solvate and/or hydrate thereof may especially be in the form of a cream, a milk, a lotion, a gel, an ointment, a pomade, a suspension of microspheres or nanospheres or lipid or polymer vesicles, impregnated pads, solutions, sprays, mousses, sticks, soaps, shampoos or washing bases.

The pharmaceutical and cosmetic compositions as described previously may also contain inert or even pharmacodynamically active additives as regards the pharmaceutical compositions, or combinations of these additives, and especially:
wetting agents;

flavour enhancers;
preserving agents such as para-hydroxybenzoic acid esters;
stabilizers;
humidity regulators;
pH regulators;
osmotic pressure modifiers;
emulsifiers;
UV-A and UV-B screening agents;
antioxidants, such as α-tocopherol, butylhydroxyanisole or butylhydroxytoluene, superoxide dismutase, ubiquinol or certain metal-chelating agents;
emollients;
moisturizers, for instance glycerol, PEG-400, thiamorpholinone and derivatives thereof, or urea;
carotenoids and especially β-carotene;
α-hydroxy acids and α-keto acids or derivatives thereof, such as lactic acid, malic acid, citric acid, glycolic acid, mandelic acid, tartaric acid, glyceric acid or ascorbic acid, and also salts, amides or esters thereof, or β-hydroxy acids or derivatives thereof, such as salicylic acid and salts, amides or esters thereof.

Needless to say, a person skilled in the art will take care to select the optional compound(s) to be added to these compositions such that the advantageous properties intrinsically associated with the present invention are not, or are not substantially, adversely affected by the envisaged addition. Moreover, in general, the same preferences as those indicated previously for the compounds of formula (I) apply mutatis mutandis to the medicaments and cosmetic and pharmaceutical compositions and to the use using the compounds of the invention.

The preparation of the active compounds of formula (I) according to the invention, and the results of the biological activity of such compounds, are given hereinbelow as illustrations and with no limiting nature.

### PROCEDURES

### Example 1

### 2-(2,4-Dioxo-1-p-tolyl-1,3-diazaspiro[4.5]dec-3-yl)-N-(1-phenylbutyl)-acetamide

### Step 1.1 2-Methyl-2-p-tolylaminobutyronitrile

### Preparation According to Scheme 3

16 ml of acetic acid are added to 1.34 g of 2-pentanone (15.5 mmol; 1 eq.) (starting material 1), followed by portionwise addition of 2 g of p-toluidine (18.6 mmol; 1.2 eq.) (starting material 2). After stirring for 30 minutes, 2.3 ml of trimethylsilyl cyanide (17 mmol; 1.1 eq.) are added, while keeping the temperature of the medium below 30° C. using an ice bath. After stirring for 4 hours at room temperature, the reaction medium is poured into 43 ml of 28% NH₄OH at 0° C. and then allowed to warm to room temperature. Ethyl acetate is added and the organic phase is extracted, dried over sodium sulfate and concentrated under vacuum. The final product is purified by chromatography on silica gel, eluting with a 90/10 heptane/ethyl acetate mixture. The product 2-methyl-2-p-tolylaminobutyronitrile is obtained in the form of an oil.

### Step 1.1 5-Methyl-5-propyl-1-p-tolylimidazolidine-2,4-dione

0.640 g (7.81 mmol; 2 eq.) of potassium cyanate is added to a solution of 0.790 g (3.9 mmol; 1.0 eq.) of 2-methyl-2-p-tolylaminobutyronitrile in 7 ml of acetic acid at 30° C. The reaction medium is heated at 60° C. for 18 hours. 10 ml of 10N HCl and then 5 ml of water are added and the reaction medium is heated at 90° C. for 7 hours and then stirred at room temperature for 3 days. The medium is poured into water and stirred for 24 hours. The precipitate is filtered off and rinsed thoroughly with water and then dried in an oven under vacuum at 40° C. The product 5-methyl-5-propyl-1-p-tolylimidazolidine-2,4-dione is obtained in the form of a white solid. Melting point=158° C.

### Step 1.1 N-(2,6-Diisopropylphenyl)-2-(4-methyl-2,5-dioxo-4-propyl-3-p-tolylimidazolidin-1-yl)acetamide

### Preparation According to Scheme 3

0.035 g (0.250 mmol; 1.1 eq.) of potassium carbonate is added to a solution of 0.056 g (0.227 mmol; 1 eq.) of 5-methyl-5-propyl-1-p-tolylimidazolidine-2,4-dione and 0.064 g (0.250 mmol; 1.1 eq.) of 2-chloro-N-(2,6-diisopropylphenyl)acetamide in 3 ml of DMF. The reaction medium is stirred at room temperature for 18 hours. 10 mg of potassium carbonate are added and stirring is continued for 24 hours. The reaction medium is poured into water and extracted with ethyl acetate. The organic phase is washed with water, dried over sodium sulfate and then concentrated to dryness. The product is precipitated by adding ethyl ether and heptane. N-(2,6-Diisopropylphenyl)-2-(4-methyl-2,5-dioxo-4-propyl-3-p-tolylimidazolidin-1-yl)acetamide is obtained in the form of a white solid. Melting point=186° C.

NMR (DMSO): 0.78 (3H, m); 1.13-1.06 (12H, m); 1.30-1.18 (1H, m); 1.35 (3H, s); 1.41-1.30 (1H, m); 1.54-1.46 (1H, m); 1.72-1.64 (1H, m); 2.34 (3H, s); 3.11-3.03 (2H, m); 4.33 (2H, m); 7.16-7.14 (2H, m); 7.16 (2H, d, J=8.10 Hz); 7.25-7.23 (1H, m); 7.28 (2H, d, J=8.10 Hz); 9.59 (1H, s)

### Preparation of the intermediate 2-chloro-N-(2,6-diisopropylphenyl)acetamide

### Synthesis According to Scheme 2

222 mL (1.59 mol) of triethylamine are added to 300 mL (1.59 mol) of 2,6-diisopropylphenylamine (Starting material 3) in 1 litre of dichloromethane. The reaction mixture is cooled to 0° C., and 127 mL (1.59 mol) of chloroacetyl chloride are then added dropwise. Once the addition is complete, the ice bath is removed and the medium is stirred for 20 minutes. It is then poured into water and extracted with dichloromethane. The organic phases are combined and washed with water. They are dried over sodium sulfate. The solvents are evaporated off. The residue is filtered through a pad of silica (eluent: dichloromethane). The filtrate is evaporated and then triturated in heptane. 2-Chloro-N-(2,6-diisopropylphenyl)acetamide is obtained in the form of a white solid.

Melting point=146-148° C.

### Example 2

Example 2 is described in Table 1 below. The compounds are synthesized according to the above procedures, replacing the starting materials 1, 2 and 3 mentioned in Examples 1, 2 and 4 with the products mentioned in

TABLE 1

| | | | | | | ¹H NMR - 400 MHz (s = |
|---|---|---|---|---|---|---|
| | | | | | | singlet, d = doublet, t = |
| | | | | | Melting | triplet, q = quartet, m = |
| | | Starting | Starting | Starting | point | multiplet, J = coupling |
| Example # | NAME | material 1 | material 2 | material 3 | (° C.) | constant) |
| 1 | N-(2,6- | pyridin-3-yl- | cyclohex- | 2,6- | 258-260 | (DMSO) 0.78 (3H, m); |
| | diisopropyl- | amine | anone | diisopro- | | 1.30-1.07 (18H, m); 1.34 |
| | phenyl)-2- | | | pylphenyl- | | (3H, s); 1.50 (1H, m); 1.70 |
| | (4-methyl- | | | amine | | (1H, m); 2.33 (3H, s); 3.08 |
| | 2,5-dioxo- | | | | | (2II, m); 4.32 (2H, m); |
| | 4-pentyl-3- | | | | | 7.16-7.14 (2H, m); 7.16 |
| | p-tolylimid- | | | | | (2H, d, J = 8.10 Hz); 7.25- |
| | azolidin-1-yl)- | | | | | 7.23 (1H, m); 7.25-7.23 |
| | acetamide | | | | | (1H, m); 7.28 (2H, d, J = |
| | | | | | | 8.10 Hz); 9.57 (1H, s) |
| 2 | N-(2,6- | 6-meth- | cyclohex- | 2,6- | 229-231 | (DMSO): 0.78 (3H, m); |
| | diiso- | oxypyridin- | anone | diisopro- | | 1.13-1.06 (12H, m); 1.30- |
| | propylphe- | 3-ylamine | | pylphenyl- | | 1.18 (1H, m); 1.35 (3H, s); |
| | nyl)-2-(4- | | | amine | | 1.41-1.30 (1H, m); 1.54- |
| | methyl-2,5- | | | | | 1.46 (1H, m); 1.72-1.64 |
| | dioxo-4- | | | | | (1H, m); 2.34 (3H, s); |
| | propyl-3-p- | | | | | 3.11-3.03 (2H, m); 4.33 |
| | tolyl- | | | | | (2H, m); 7.16-7.14 (2H, |
| | imidazol- | | | | | m); 7.16 (2H, d, J = 8.10 |
| | idin-1- | | | | | Hz); 7.25-7.23 (1H, m); |
| | yl)acet- | | | | | 7.28 (2H, d, J = 8.10 Hz); |
| | amide | | | | | 9.59 (1H, s) |

All the NMR (nuclear magnetic resonance) spectra are in accordance with the proposed structures. The chemical shifts are expressed in parts per million. The internal reference is tetramethylsilane. The following abbreviations are used: CDCl₃=deuterated chloroform, DMSO=deuterated dimethyl sulfoxide

### Example 3

### Biological Tests

The compounds of formula (I) according to the invention were subjected to a test for evaluating their inhibitory activity towards the enzyme ACAT-1, inspired by the following publication: “Identification of ACAT1- and ACAT2-specific inhibitors using a novel, cell based fluorescence assay: individual ACAT uniqueness”, J. Lipid. Res. (2004) vol. 45, pages 378-386.

The principle of this test is based on the use of NBD-cholesterol, a cholesterol analogue whose fluorescence depends on its environment. When this molecule is in a polar environment, it is weakly fluorescent, whereas in a non-polar environment, it is strongly fluorescent. Free NBD-cholesterol becomes inserted in cell membranes and is weakly fluorescent in this polar environment. When NBD-cholesterol is esterified with ACAT, the NBD-cholesterol ester enters non-polar lipid droplets and is then strongly fluorescent.

The method below is applied: HepG2 cells are incubated in the presence of NBD-cholesterol (1 μg/ml) and of the test compound of formula (I) in black transparent-bottomed 96-well plates, at a rate of 30 000 cells per well. After incubation for 6 hours at 37° C. under 5% CO₂, the medium is removed by turning upside-down and the cells are washed with twice 100 μl of PBS. After addition of 50 μl of lysis buffer (10 mM NaPO₄, 1% Igepal), the plates are shaken for 5 minutes and the fluorescence is read (excitation at 490 nm, emission at 540 nm) on a Fusion machine (Perkin-Elmer). By way of illustration, an IC₅₀ of 9 nM is obtained for compound (1) and an IC₅₀ of 3 nM is obtained for compound (2).

### Example 4

### Formulations

Various formulations containing the compounds according to the invention are given below.

### A—Oral Route

### (a) 0.2 g Tablet

| | |
|---|---|
| Compound 1 | 0.01 g |
| Starch | 0.114 g |
| Dicalcium phosphate | 0.020 g |
| Silica | 0.020 g |
| Lactose | 0.030 g |
| Talc | 0.010 g |
| Magnesium stearate | 0.005 g |

### (b) Drinkable Suspension in 5 ml Vials

| Compound 2 | 0.001 | g |
|---|---|---|
| Glycerol | 0.500 | g |
| 70% Sorbitol | 0.500 | g |
| Sodium saccharinate | 0.010 | g |
| Methyl para-hydroxybenzoate | 0.040 | g |
| Flavouring | qs | |
| Purified water | qs 5 | ml |

### B—Topical Route

### (a) Ointment

| | | |
|---|---|---|
| Compound 1 | 0.300 | g |
| White petroleum jelly codex | qs 100 | g |

### (d) Lotion

| | |
|---|---|
| Compound 2 | 0.100 g |
| Polyethylene glycol (PEG 400) | 69.900 g |
| 95% Ethanol | 30.000 g |

### (e) Hydrophobic Ointment

| | | |
|---|---|---|
| Compound 2 | 0.300 | g |
| Isopropyl myristate | 36.400 | g |
| Silicone oil (Rhodorsil 47 V 300) | 36.400 | g |
| Beeswax | 13.600 | g |
| Silicone oil (Abil 300 000 cSt) | qs 100 | g |

### (f) Nonionic Oil-in-Water Cream

| | | |
|---|---|---|
| Compound 1 | 1.000 | g |
| Cetyl alcohol | 4.000 | g |
| Glyceryl monostearate | 2.500 | g |
| PEG 50 stearate | 2.500 | g |
| Shea butter | 9.200 | g |
| Propylene glycol | 2.000 | g |
| Methyl para-hydroxybenzoate | 0.075 | g |
| Propyl para-hydroxybenzoate | 0.075 | g |
| Sterile demineralized water | qs 100 | g |

1. A compound of formula (I): 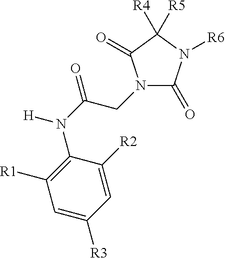
in which:
R₁ represents a group C₁₋₆ alkyl, C₃₋₇ cycloalkyl, C₁₋₆ alkyloxy, C₁₋₆ fluoroalkyl or C₁₋₆ fluoroalkyloxy or a group —(CH₂)ₙ—C₃₋₇ cycloalkyl,
R₂ and R₃ are identical or different and represent hydrogen, chlorine, fluorine, bromine or iodine atom or a group C₁₋₆ alkyl, C₃₋₇ cycloalkyl, C₁₋₆ alkyloxy, C₁₋₆ fluoroalkyl or C₁₋₆ fluoroalkyloxy or a group —(CH₂)ₙ—C₃₋₇ cycloalkyl,
R₄ and R₅ are different from each other and individually represent:
either a hydrogen atom,
or a group C₁₋₆ alkyl optionally substituted with one to three groups Rₐ,
or a group C₃₋₇ cycloalkyl or a group —(CH₂)ₙ—C₃₋₇ cycloalkyl,
R₆ represents a group selected from the group consisting of:
an unsubstituted phenyl group or a phenyl group substituted with one, two or three identical or different substituents selected from the group consisting of fluorine, chlorine and bromine atoms, and groups C₁₋₄ alkyl, C₁₋₄ alkylthio, trifluoromethyl, hydroxymethyl, mono-, di- and trifluoromethoxy, C₁₋₄ alkyloxy, hydroxyl, COOR_{b}, CN, phenoxy, benzyloxy, phenyl, 2-pyridyl, 3-pyridyl and 4-pyridyl,
a linear or branched group C₂₋₁₂ alkyl optionally substituted with one or more hydroxyl groups or fluorine atoms,
a group C₃₋₇cycloalkyl or a group —(CH₂)ₚ—C₃₋₇cycloalkyl,
a group —(CH₂)ₙ-aryl in which n is equal to 1, 2 or 3 and the aryl group is optionally substituted with one or more groups Rₐ,
a group —(CH₂)ₙ—Ar with n equal to 1, 2 or 3 and Ar representing an unsubstituted phenyl or unsubstituted alkyl group, or a phenyl or naphthyl group substituted with one to three identical or different substituents selected from the group consisting of fluorine, chlorine, iodine or bromine atoms and groups C₁₋₆alkyl, hydroxymethyl, mono-, di- or trifluoromethyl, hydroxy, phenyl, 2-pyridyl, 3-pyridyl or 4-pyridyl, C₁₋₆alkyloxy, phenoxy, benzyloxy, and mono-, di- or trifluoromethoxy,
Rₐ represents either a hydrogen, fluorine, chlorine or bromine atom or a group C₁₋₆ alkyl, C₃₋₇ cycloalkyl, C₁₋₆ alkyloxy, C₁₋₆ alkylthio, C₁₋₆ fluoroalkyl or C₁₋₆ fluoroalkyloxy, or a group —(CH₂)ₙ—C₃₋₇ cycloalkyl, OH, COOR_{b} or CN,
R_{b} represents a group C₁₋₆ alkyl, C₃₋₇ cycloalkyl or —(CH₂)ₙ—C₃₋₇ cycloalkyl,
n is an integer equal to 1, 2 or 3,
and also the pharmaceutically acceptable salts, solvates or hydrates thereof and the conformers or rotamers thereof.

2. The compound according to claim 1, wherein:
R₁ represents a group C₁₋₆ alkyl, C₃₋₇ cycloalkyl, C₁₋₆ alkyloxy, C₁₋₆ fluoroalkyl or C₁₋₆ fluoroalkyloxy or more favourably a chlorine, methyl, ethyl, isopropyl, tert-butyl, cyclopropyl or CH₂-cyclopropyl group,
R₂ represents a hydrogen, chlorine, fluorine or bromine atom or a methyl, ethyl, isopropyl or CH₂-cyclopropyl group,
R₃ represents a hydrogen atom.

3. The compound according to claim 2, wherein R₁ represents a chlorine, methyl, ethyl, isopropyl, tert-butyl, cyclopropyl or CH₂-cyclopropyl.

4. The compound according to claim 3, wherein R₁ represents a methyl, ethyl, propyl or isopropyl group.

5. The compound according to claim 1, wherein the groups R₄ and R₅ are different and represent a nitrogen atom or a methyl, ethyl, propyl, butyl, isopropyl, cyclopropyl, cyclobutyl or CH₂-cyclopropyl group.

6. The compound according to claim 5, wherein R₄ is a methyl and R₅ is an ethyl or a propyl.

7. The compound according to claim 1, wherein the group R₆ represents an unsubstituted phenyl group or a phenyl group substituted, in the meta or para position, with a chlorine or fluorine atom, methyl or methoxy.

8. The compound according to claim 1, selected from the group of compounds below, and pharmaceutically acceptable salts, solvates, hydrates, conformers and rotamers thereof:
N-(2,6-diisopropylphenyl)-2-(4-methyl-2,5-dioxo-4-propyl-3-p-tolylimidazolidin-1-yl)-acetamide;
N-(2,6-diisopropylphenyl)-2-(4-methyl-2,5-dioxo-4-pentyl-3-p-tolylimidazolidin-1-yl)-acetamide;
N-(2,6-diethylphenyl)-2-(4-methyl-2,5-dioxo-4-propyl-3-p-tolylimidazolidin-1-yl)acetamide;
2-[3-(4-chlorophenyl)-4-methyl-2,5-dioxo-4-propylimidazolidin-1-yl]-N-(2,6-diisopropyl-phenyl)acetamide; and
N-(2,6-diisopropyl-phenyl)-2-(4-methoxymethyl-4-methyl-2,5-dioxo-3-p-tolylimidazolidin-1-yl)acetamide.

9. The compound according to claim 1, as a medicament.

10. A pharmaceutical composition comprising, in a physiologically acceptable support, at least one compound according to claim 1.

11. The composition according to claim 10, wherein the concentration of compound according to claim 1 is between 0.001% and 10% by weight relative to the total weight of the composition.

12. The composition according to claim 11, wherein the concentration of compound according to claim 1 is between 0.01% and 5% by weight relative to the total weight of the composition.

13. A cosmetic composition, comprising, in a physiologically acceptable support, at least one compound according to claim 1.

14. A composition according to claim 1, wherein it is in a form suitable for topical application.

15. The composition according to claim 14, wherein it is in the form of a cream, a milk, a lotion, a gel, an ointment, a pomade, a suspension of microspheres or nanospheres or lipid or polymer vesicles, an impregnated pad, a solution, a spray, a mousse, a stick, a soap, a shampoo or a washing base.

16. A cosmetic method, the method comprising administering a composition as defined in claim 13 to an individual subject in need thereof, for body or hair hygiene.

17. A method of making a medicament, the method comprising making the medicament so that it comprises a compound according to claim 1, wherein the compound is present in an amount effective to treat an indication selected from the group consisting of a sebaceous gland disorder, an ocular pathology, hypercholesterolaemia, arteriosclerosis and Alzheimer's disease.

18. A method of making a medicament for treating acne, the method comprising making the medicament so that it comprises an effective amount of a compound according to claim 1.

19. The method of claim 17, wherein the sebaceous gland disorder is hyperseborrhoea, acne seborrhoeic dermatitis or atopic dermatitis.

20. The method of claim 17, wherein the occular pathology is blepharitis or neibomitis.
